(19) United States
(12) Patent Application Publication
(10) Pub. No.: US 2023/0072384 A1
(43) Pub. Date: Mar. 9, 2023

(54) Telescopic Impact Sperm Collector

(71) Applicant: SHENZHEN SHAKI INDUSTRIAL CO., LTD, Shenzhen (CN)

(72) Inventor: Xiangru Chen, Shenzhen (CN)

(21) Appl. No.: 17/470,004

(22) Filed: Sep. 9, 2021

(51) Int. Cl. A61B 10/00; A61H 19/00

(52) U.S. Cl. CPC A61B 10/0058; A61H 19/32; A61H 2201/12; A61H 2201/0107; A61H 2201/1418

(57) ABSTRACT

A telescopic impact sperm collector includes an outer cup body, a soft cup body, and an inner cup body, a transmission mechanism and a drive mechanism which are installed in the outer cup body. The soft cup body has a massage cavity with a front opening; both ends of the soft cup body are connected to a cup mouth of the outer cup body and the inner cup body respectively; the drive mechanism includes a brushless motor; the transmission mechanism includes a mandrel and a cam; the mandrel has an end hinged to a rear end of the inner cup body and the other end transmissively connected to the brushless motor through the cam; the inner cup body can be driven by the brushless motor to slide back and forth in the outer cup body.

### FIELD OF THE INVENTION

The present invention relates to the field of sperm collection, and more particularly to a telescopic impact sperm collector.

### BACKGROUND OF THE INVENTION

With the continuous economic and social development, the pressure of life is increasing, thus leading to a decline in male sperm motility, and the problem of infertility becomes increasingly more prominent. During medical treatments in hospitals, it is often necessary to obtain sperm and perform various tests on sperm in a short period of time. However, the poor sperm collection environment of the outpatient clinic and the large psychological barrier of the patients have great impacts on the sperm collection efficiency. The sperm collector is one of the necessary equipment for male medical treatment and testing, and the common sperm collector is in contact with human body through a soft cup body, and an inner cup body of the sperm collector transmits the generated driving forces to the soft cup body to realize the massage action in order to collect sperms.

The power part of the sperm collector is generally an ordinary motor. To improve the operating stability of the sperm collector, the power of the ordinary motor connected to the mechanism of the inner cup body is designed with a decelerator or a gear box in order to improve the rotation precision, and make the operation of the sperm collector more stable, so as to improve the user’s feeling and the sperm collection efficiency. However, the use of the decelerator or gear box will result in increased total volume of the sperm collector, high production cost, low strength, slow telescopic frequency, and large noise. Therefore, it is necessary to improve the prior art.

### SUMMARY OF THE INVENTION

In view of the aforementioned drawbacks of the prior art, the present invention provides a telescopic impact sperm collector that adopts a brushless motor as a power source and uses a cam and a mandrel replace the decelerator for transmission, so as to simplify the overall structure, and reduce the volume and manufacturing cost of the sperm collector, and the invention also has the advantages of simple structure, powerful force, low noise and high and stable telescopic frequency.

To overcome the drawbacks of the prior art, the present invention discloses a technical solution and provide a telescopic impact sperm collector, and the sperm collector includes an outer cup body, a soft cup body, and an inner cup body, a transmission mechanism and a drive mechanism which are disposed in the outer cup body. The soft cup body has a massage cavity with a front opening; both ends of the soft cup body are connected to a cup mouth of the outer cup body and the inner cup body respectively; the drive mechanism includes a brushless motor; the transmission mechanism includes a mandrel and a cam; the mandrel has an end hinged to a rear end of the inner cup body and the other end transmissively connected to the brushless motor through the cam; and the inner cup body is driven by the brushless motor and can slide back and forth in the outer cup body.

The additional structure of the aforementioned technical solution may include the following solutions:

In a specific embodiment, the telescopic impact sperm collector further includes a guide mechanism, and both ends of the guide mechanism have a guide rod connected to an inner side of the outer cup body; and a side of the inner cup body is movably connected to the guide rod; and a side of the inner cup body is movably connected to the guide rod.

Further, both ends of the guide rod have a fastener; and an inner side of the outer cup body has a snap column detachably connected to the fastener.

Further, the inner cup body has a sliding sleeve disposed on an outer side thereof and movably sheathed on the guide rod.

In a specific embodiment, the inner cup body has two corresponding connecting seats disposed at a rear end of the inner cup body, and each of the two connecting seats has a shaft hole formed on the connecting seat; the end of the mandrel corresponding to the inner cup body has a shaft; the end of the mandrel corresponding to the inner cup body is disposed between the two connecting seats and rotatably connected to the shaft hole through the shaft.

In a specific embodiment, the end of the mandrel corresponding to the brushless motor is embedded with a deep groove ball bearing; the cam has an eccentric column formed on the cam and rotatably connected to the mandrel through the deep groove ball bearing.

In a specific embodiment, the outer cup body has a snap socket formed at the cup mouth; the exterior of the snap socket is covered by a protective cover; and the interior of the snap cover has a protective case.

In a specific embodiment, the inner cup body has an insertion cavity with a front opening, and a rear end of the soft cup body is snapped into the insertion cavity.

In a specific embodiment, the exterior of the outer cup body is sheathed with a hollow decorative shell.

In a specific embodiment, the telescopic impact sperm collector further includes a charging base and a power supply module; the power supply module includes a lithium battery installed on an inner side of the outer cup body and a charging contact formed at the bottom of the outer cup body; the bottom of the outer cup body is plugged onto the charging base, so that the charging contact is conductively contacted with the charging base.

The present invention has the following advantageous effects:

The invention adopts the brushless motor to replace the traditional motor, omits the use of the decelerator in the traditional sperm collector, and uses the mandrel and the cam for the transmission instead, so as to achieve the effects of simplifying the overall structure and reducing the volume and manufacturing cost of the sperm collector. Of course, the invention also has the advantages of simple structure, powerful force, low noise and high and stable telescopic frequency that can greatly improve the user’s feeling and the sperm collection efficiency.

### BRIEF DESCRIPTION OF THE DRAWINGS

FIG. 1 is a schematic view showing the overall structure of the present invention;

FIG. 2 is an exploded view of the present invention;

FIG. 3 is a first schematic view of a partial structure of the present invention; and

FIG. 4 is a second schematic view of a partial structure of the present invention;

### DESCRIPTION OF THE PREFERRED EMBODIMENTS

The technical contents of the present invention will become apparent with the detailed description of preferred embodiments accompanied with the illustration of related drawings as follows. It is intended that the embodiments and figures disclosed herein are to be considered illustrative rather than restrictive.

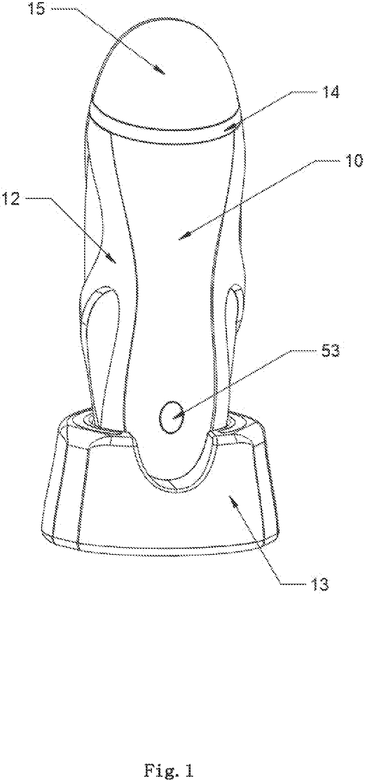

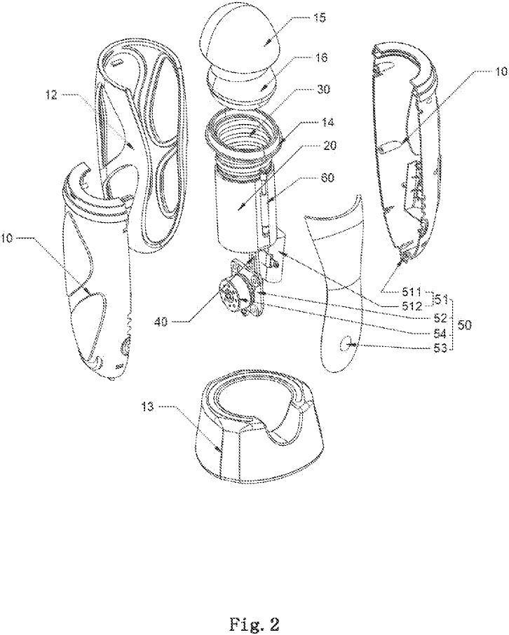

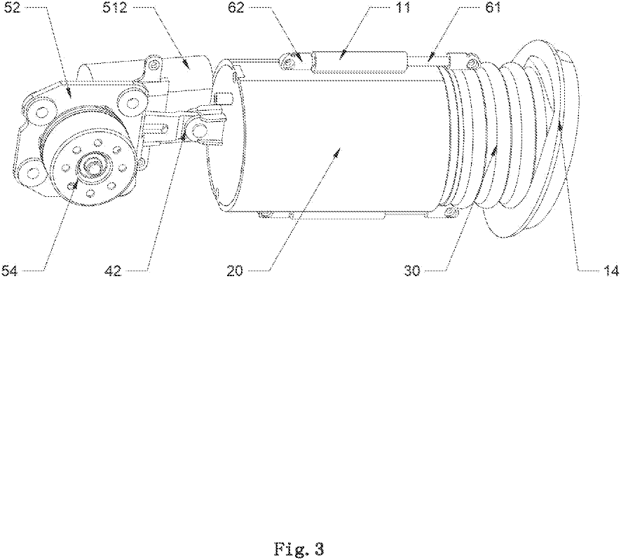

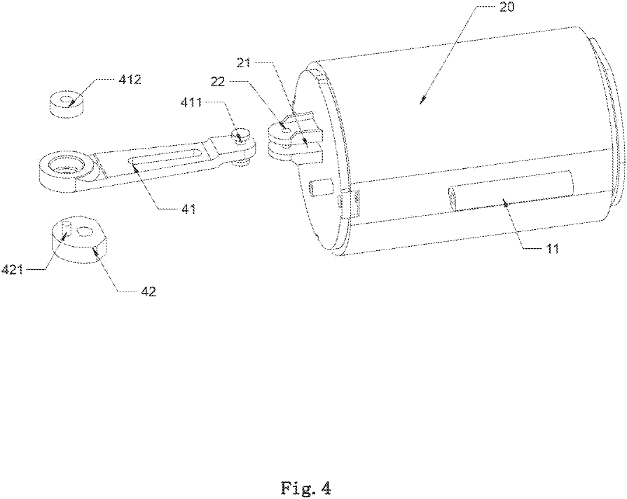

With reference to FIGS. 1 to 4 for a telescopic impact sperm collector of the present invention, the telescopic impact sperm collection includes an outer cup body 10, a soft cup body 30, and an inner cup body 20, a transmission mechanism 40 and a drive mechanism 50 which are disposed in the outer cup body 10. The soft cup body 30 has a massage cavity with a front opening; both ends of the soft cup body 30 are connected to a cup mouth of the outer cup body 10 and the inner cup body 20 respectively; the drive mechanism 50 includes a brushless motor 54; the transmission mechanism 40 includes a mandrel 41 and a cam 42; the mandrel 41 has an end hinged to a rear end of the inner cup body 20 and the other end transmissively connected to the brushless motor 54 through the cam 42 (specifically connected to an output shaft of the brushless motor 54); the inner cup body 20 is driven by the brushless motor 54 and capable of sliding back and forth in the outer cup body 10, so as to drive the rear end of the soft cup body 30 to move synchronously.

The telescopic impact sperm collector further includes a guide mechanism 60; the guide mechanism 60 has a guide rod 61 with both ends connected to an inner side of the outer cup body 10; a side of the inner cup body 20 is movably connected to the guide rod 61 to facilitate the positioning of the inner cup body 20 and prevent it from being deviated from the predetermined moving path. In this embodiment, to make the motion of inner cup body 20 more stable, the guide rod 61 has two and only two guide rods 61 movably connected to both sides of the inner cup body 20.

Specifically, both ends of the guide rod 61 have a fastener 62 separately; the inner side of the outer cup body 10 has a snap column (not shown in the figure) detachably coupled to the fastener 62. With the connection of the snap column and the fastener 62, the guide rod 61 can be fixed securely to prevent it from being loosen or falling off, and to ensure the stability for the motion of the inner cup body 20.

Specifically, the outer side of the inner cup body 20 further has a sliding sleeve 11 movably sheathed on the guide rod 61. In other words, the inner cup body 20 is movably connected to the guide rod 61 through the sliding sleeve 11, so that when the sliding sleeve 11 is moving, it will always move on the guide rod 61 to further improve the stability of the movement of the inner cup body 20.

Two corresponding connecting seats 21 are disposed at a rear end of the inner cup body 20, and each of the two connecting seats 21 has a shaft hole 22; the end of the mandrel 41 corresponding to the inner cup body 20 has a shaft 411; the end of the mandrel 41 corresponding to the inner cup body 20 is disposed between the two connecting seats 21 and rotatably connected to the shaft hole 22 through the shaft 411. By the installation of the connecting seats 21 and the shaft 411, the stability of the connection between the mandrel 41 and the inner cup body 20 can be guaranteed, so that the mandrel 41 can drive the inner cup body 20 to move in a better way.

The end of the mandrel 41 corresponding to the brushless motor 54 is embedded with a deep groove ball bearing 412; the cam 42 has an eccentric column 421, and the eccentric column 421 and the mandrel 41 are rotatably connected through the deep groove ball bearing 412. With the installation of the mandrel 41 and the deep groove ball bearing 412 rotatably connected to the eccentric column 421, direct contact friction between the cam 42 and the mandrel 41 can be avoided, so that when the cam 42 is rotated, the inner ring of the deep groove ball bearing 412 can be rotated synchronously, and then the deep groove ball bearing 412 is used to drive the motion of the mandrel 41, so as to achieve a more stable driving effect, and improve the service life of the cam 42 and the mandrel 41 to some extent.

The outer cup body 10 has a snap socket 14 formed at the position of a cup mouth of the outer cup body 10; the exterior of the snap socket 14 is snapped and covered with a protective cover 15; the interior of the snap socket 14 is sheathed with a protective case 16. The protective cover 15 can be used to protect the interior space of the inner cup body 20 and the soft cup body 30, and the protective case 16 can be used for storing sperm.

The inner cup body 20 has an insertion cavity with a front opening; the rear end of the soft cup body 30 is snapped into the insertion cavity. With the aforementioned installation, the inner cup body 20 can be moved in a better and more stable manner to drive the soft cup body 30 to move synchronously.

In this embodiment, an inner wall of the massage cavity has massage bumps capable of providing a better massaging effect, and the soft cup body 30 is preferably made of silicone. Of course, a person having ordinary skill in the art should know that the soft cup body 30 can also be made of other soft materials.

The exterior of the outer cup body 10 is sheathed with a hollow decorative shell 12 to improve the aesthetic appearance of the product.

The telescopic impact sperm collector further includes a charging base 13 and a power supply module 51, and the power supply module 51 includes a lithium battery 512 installed on an inner side of the outer cup body 10 and a charging contact 511 installed at the bottom of the outer cup body 10, and the bottom of the outer cup body 10 can be plugged onto the charging base 13, so that the charging contact 511 and the charging base 13 are electrically contacted to facilitate the charging operation. Preferably, the lithium battery 512 is a rechargeable battery.

Of course, the telescopic impact sperm collector can further includes a circuit control module 52 and a button operation module 53. The button operation module 53 includes a button installed on a surface of the outer cup body 10, and provided for the control of turning on or off the sperm collector as well as adjusting the massage frequency of the sperm collector.

The brushless motor 54, the button and the lithium battery 512 are electrically connected to the circuit control module 52.

The present invention adopts the brushless motor to replace the traditional motor, omits the decelerator installed in the traditional sperm collector, and uses the mandrel and cam for transmission instead to simplify the overall structure and reduce the volume and manufacturing cost of the sperm collector. Of course, the invention further has the advantages of simple structure, powerful force, low noise and high and stable telescopic frequency, which can greatly improve the user’s feeling and the sperm collection efficiency. Since the operation efficiency, the low-speed torque, the speed accuracy, etc. of the brushless motor are better than the frequency converter of any control technology, therefore the method of using the brushless motor can make the operation of the sperm collector more stable, and the transmission structure adopting the mandrel and the cam together with the guide rod can realize the directional telescopic action of the inner cup body relative to the outer cup body to improve the stability. By sending a command signal to the circuit control module through the button, the circuit control module will drive the brushless motor to operate after receiving the command signal, so as to drive the cam to rotate, and after rotating, the cam will drive the mandrel to have a reciprocating motion, so that the inner cup body together with the guide rod are moved back and forth by the mandrel to produce a reciprocating motion, and drive the soft cup body to complete a telescopic motion for sperm collection. With the aforementioned design, the invention has the advantages of simple overall structure, stable operation, improved users’ feeling and high sperm collection efficiency.

The specific principle of the application of the present invention is described in order to understand the technical characteristics of the invention:

Firstly, after the sperm collector is turned on by pressing a button, the brushless motor 54 drives the mandrel 41 to perform a reciprocating motion through the cam 42, and the mandrel 41 drives the rear ends of the inner cup body 20 and the soft cup body 30 to perform a reciprocating motion along the front and back direction. When an object to be massaged is stretched from the opening into the massage cavity, the soft cup body 30 is moving in the front and back direction to massage the object to be massaged, and massage bumps formed on the inner wall of the massage cavity will also massage the massaging object to give a good massage feeling to users, so as to improve the sperm collection efficiency to some extent.

While the invention has been described by way of example and in terms of a preferred embodiment, it is to be understood that the invention is not limited thereto. To the contrary, it is intended to cover various modifications and similar arrangements and procedures, and the scope of the appended claims therefore should be accorded the broadest interpretation so as to encompass all such modifications and similar arrangements and procedures.

What is claimed is:

1. A telescopic impact sperm collector, comprising: an outer cup body, a soft cup body, and an inner cup body, a transmission mechanism and a drive mechanism which are disposed in the outer cup body; the soft cup body having a massage cavity with a front opening; both ends of the soft cup body being coupled to a cup mouth of the outer cup body and the inner cup body respectively; the drive mechanism comprising a brushless motor; the transmission mechanism comprising a mandrel and a cam; the mandrel having an end hinged to a rear end of the inner cup body and the other end transmissively coupled to the brushless motor through the cam; and the inner cup body being driven by the brushless motor and capable of sliding back and forth in the outer cup body.

2. The telescopic impact sperm collector according to claim 1, further comprising a guide mechanism; and both ends of the guide mechanism having a guide rod coupled to an inner side of the outer cup body; and a side of the inner cup body being movably coupled to the guide rod.

3. The telescopic impact sperm collector according to claim 2, wherein both ends of the guide rod have a fastener; and an inner side of the outer cup body has a snap column detachably coupled to the fastener.

4. The telescopic impact sperm collector according to claim 2, wherein the inner cup body has a sliding sleeve disposed on an outer side thereof and movably sheathed on the guide rod.

5. The telescopic impact sperm collector according to claim 1, wherein the inner cup body has two corresponding connecting seats disposed at a rear end thereof, and each of the two connecting seats has a shaft hole formed thereon separately; the end of the mandrel corresponding to the inner cup body has a shaft; the end of the mandrel corresponding to the inner cup body is disposed between the two connecting seats and rotatably coupled to the shaft hole through the shaft.

6. The telescopic impact sperm collector according to claim 1, wherein the end of the mandrel corresponding to the brushless motor is embedded with a deep groove ball bearing; the cam has an eccentric column formed thereon and rotatably coupled to the mandrel through the deep groove ball bearing.

7. The telescopic impact sperm collector according to claim 1, wherein the outer cup body has a snap socket formed at the cup mouth; the exterior of the snap socket is covered by a protective cover; and the interior of the snap cover has a protective case.

8. The telescopic impact sperm collector according to claim 1, wherein the inner cup body has an insertion cavity with a front opening formed thereon, and a rear end of the soft cup body is snapped into the insertion cavity.

9. The telescopic impact sperm collector according to claim 1, wherein the exterior of the outer cup body is sheathed with a hollow decorative shell.

10. The telescopic impact sperm collector according to claim 1, further comprising a charging base and a power supply module; the power supply module comprising a lithium battery installed on an inner side of the outer cup body and a charging contact formed at the bottom of the outer cup body; the bottom of the outer cup body being plugged onto the charging base, so that the charging contact is conductively contacted with the charging base.
